(19) United States
(12) Patent Application Publication
(10) Pub. No.: US 2025/0033065 A1
(43) Pub. Date: Jan. 30, 2025

(54) AIR PURIFICATION FILTER DEVICE USING X-RAY IONIZER

(71) Applicant: Cobalt Technology Inc., Daejeon-si (KR)

(72) Inventor: Jeong Seok BAEK, Seoul (KR)

(73) Assignee: Cobalt Technology Inc., Daejeon-si (KR)

(21) Appl. No.: 18/493,476

(22) Filed: Oct. 24, 2023

(30) Foreign Application Priority Data
Jul. 24, 2023 (KR) 10-2023-0095781

(51) Int. Cl. B03C 3/155; B01D 46/00; B01D 46/42; B01D 46/79; B03C 3/145; B03C 3/38

(52) U.S. Cl. CPC B03C 3/155; B01D 46/0032; B01D 46/0038; B01D 46/4209; B01D 46/79; B03C 3/145; B03C 3/383

(57) ABSTRACT

Provided is an air purification filter device using an X-ray ionizer that cleanly filters contaminated air and discharges cleaned air by supplying the contaminated air such as bad odors and fine dust from restaurants, factories, livestock farms, and manufacturing facilities to the device, charging positive or negative ions to contaminated air using an X-ray ionizer, and collecting contaminated air by static electricity in the baffle filter and metal mesh filter using the Coanda Effect.

### TECHNICAL FIELD

The present invention relates to an air purification filter device using an X-ray ionizer, and more specifically, relates to an air purification filter device using an X-ray ionizer that cleanly filters contaminated air and discharges cleaned air by supplying the contaminated air such as bad odors and fine dust from restaurants, factories, livestock farms, and manufacturing facilities to the device, charging positive or negative ions to contaminated air using an X-ray ionizer, and collecting contaminated air by static electricity in the baffle filter and metal mesh filter using the Coanda Effect.

### BACKGROUND ART

Fine dust is small, so it has the characteristic of penetrating deep into the alveoli without being caught in the mouth or nasal passages when the human body breathes. It also causes many difficulties in securing visibility due to optical properties such as refraction and scattering of light.

Additionally, fine dust contains many harmful substances, so fine dust that penetrates the lungs remains in the lungs and can cause very serious respiratory diseases such as pneumonia, lung cancer, and bronchitis.

This type of fine dust is commonly emitted along with bad odors in restaurants, factories, livestock facilities (pig houses, chicken houses, etc.), and manufacturing facilities.

The fine dust emission sources emit fine dust and odor to the outside, and the fine dust and odor inside the emission sources can also have a negative impact on the health of workers.

Accordingly, various air purification devices are becoming popular to purify the air by removing fine dust, bad odors, and various foreign substances.

These air purification devices purify the air by supplying polluted air to a filter module and require periodic cleaning or replacement of the filter module. However, management such as cleaning and replacement of such filter modules is not carried out well in practice.

For example, most air purifiers currently in use are equipped with HEPA filters that can remove more than 99.97% of particles as large as 0.3 μm. HEPA filters are very effective in filtering out fine dust and purifying the air, but it is impossible to shake off or wash off the fine dust collected in the filter and reuse it, so it is necessary to discard it after a certain period of use and replace it with a new filter. In this process, costs are incurred regularly, and if replacement is not done at the appropriate time, the contaminant removal performance of the air purifier can rapidly deteriorate, worsening the user's health.

A conventional technology developed to solve this problem is disclosed in Korean Patent Registration No. 10-2200897.

However, this conventional technology allows continuous reuse through cleaning of the removal part, resolving the economic burden on consumers. However, if the process of completely drying the collection plate after cleaning it with water is not carried out, electrical accidents such as sparks may cause device failure, electric shock, or fire.

Therefore, it is necessary to develop technology for the air purification filter device that can be used safely even without any special measures and can be used without the financial burden of replacing the filter module.

### Prior Art Document

(Patent Document 1) Korean Patent Registration No. 10-2200897

### DISCLOSURE

### Technical Problem

The present invention, which was devised to solve this problem, relates to an air purification filter device using an X-ray ionizer, and more specifically, relates to an air purification filter device using an X-ray ionizer that cleanly filters contaminated air and discharges cleaned air by supplying the contaminated air such as bad odors and fine dust from restaurants, factories, livestock farms, and manufacturing facilities to the device, charging positive or negative ions to contaminated air using an X-ray ionizer, and collecting contaminated air by static electricity in the baffle filter and metal mesh filter using the Coanda Effect.

### Technical Solution

In order to achieve the above object, the present invention provides an air purification filter device using an X-ray ionizer comprising: A first chamber (110) connected to a blower and supplied with contaminated air;

An ionizer unit (120) comprising an ionizer output means (121) formed in a selected portion of the first chamber (110) and providing ions to the contaminated air supplied to the first chamber (110), and
an ionizer body (122) that provides a light source to the ionizer output means (121);

A filter unit (130) comprising a baffle filter (131) formed of a plurality of pillars with a semicircular cross-section and providing a surface for adsorbing fine dust and odor particles from contaminated air charged with positive or negative ions through the ionizer unit (120), and
a mesh filter (132) formed of a metal mesh for adsorbing fine dust and odor particles that have passed through the baffle filter (131);

A cleaning unit (140) comprising a baffle filter cleaning means (141) for cleaning particles adsorbed on the surface of the baffle filter (131) by spraying a cleaning solution onto the baffle filter (131), and
a mesh filter cleaning means (142) that sprays a cleaning solution on the mesh filter (132) to clean particles adsorbed on the surface of the mesh filter (132); and

A second chamber (150) comprising a rear filter (151) that filters foreign substances in the air that have passed through the filter unit (130).

At this time, the ionizer unit (120) may have a voltage of 3 to 30 kV.

Additionally, the filter unit (130) may be configured to apply the Coanda Effect to the baffle filter (131).

In addition, the filter unit (130) may be configured to provide a ground connection to the baffle filter (131) and the mesh filter (132).

In addition, the cleaning unit (140) comprises a filter status check sensor (143) that checks the status of the baffle filter (131) and the mesh filter (132), and
a cleaning control means (144) that analyzes the status of the baffle filter (131) and mesh filter (132) confirmed through the filter status check sensor (143), and controls cleaning through the baffle filter cleaning means (141) and the mesh filter cleaning means (142) when the contamination level exceeds the pre-entered level.

### Advantageous Effect

The present invention can provide an air purification filter device using an X-ray ionizer that cleanly filters contaminated air and discharges cleaned air by supplying the contaminated air such as bad odors and fine dust from restaurants, factories, livestock farms, and manufacturing facilities to the device, charging positive or negative ions to contaminated air using an X-ray ionizer, and collecting contaminated air by static electricity in the baffle filter and metal mesh filter using the Coanda Effect.

The photo ionizer using X-rays of the present invention does not require airflow supply, does not generate foreign substances, and maintenance of the ionizer part can be easily performed.

In addition, the baffle filter and mesh filter are grounded, so they can effectively absorb fine dust and odor particles in contaminated air.

The baffle filter can improve the adsorption rate of fine dust and odor particles by using the Coanda effect, and contaminated air can be purified more effectively.

In addition, the baffle filter cleaning means and mesh filter cleaning means automatically and periodically clean the baffle filter and mesh filter to optimally maintain contamination level of filter surface, thereby preventing a decrease in adsorption and purification efficiency that may occur due to contamination of the filter.

### BRIEF EXPLANATION OF DRAWINGS

FIG. 1 is an embodiment of an air purification filter device using the X-Ray Ionizer of the present invention.

FIG. 2 is an embodiment of the air purification filter device of the present invention.

FIG. 3 is an example of flow analysis of the air purification filter device of the present invention.

FIG. 4 shows an example of the cleaning procedure of the cleaning unit of the present invention.

### MODE FOR THE INVENTION

Hereinafter, the present invention will be described in more detail with reference to the attached drawings. Prior to this, the terms or words used in this specification and claims should not be construed as limited to their usual or dictionary meanings, and the inventor should appropriately define the concept of terms in order to explain his or her invention in the best way. Based on the principle of definability, it must be interpreted with meaning and concept consistent with the technical idea of the present invention. In addition, if there is no other definition in the technical and scientific terms used, they have meanings commonly understood by those skilled in the art to which this invention pertains, and the gist of the present invention is summarized in the following description and accompanying drawings. Descriptions of known functions and configurations that may be unnecessarily obscure are omitted. The drawings introduced below are provided as examples so that the idea of the present invention can be sufficiently conveyed to those skilled in the art. Accordingly, the present invention is not limited to the drawings presented below and may be embodied in other forms. Additionally, like reference numerals refer to like elements throughout the specification. It should be noted that like elements in the drawings are represented by like symbols wherever possible.

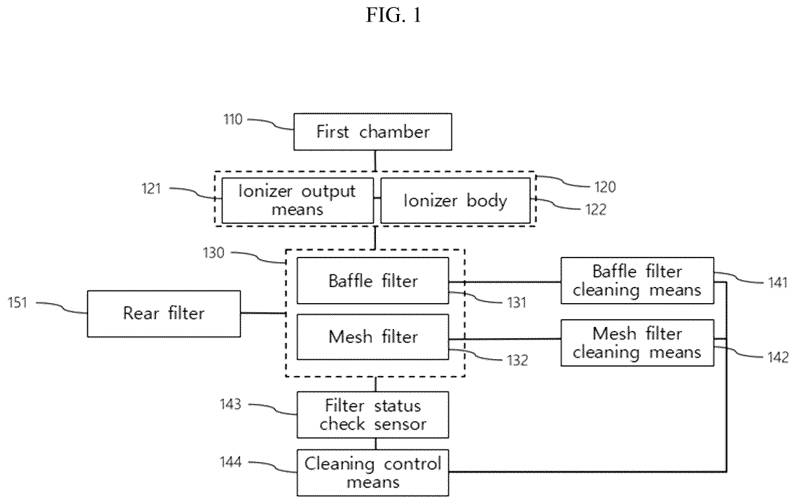

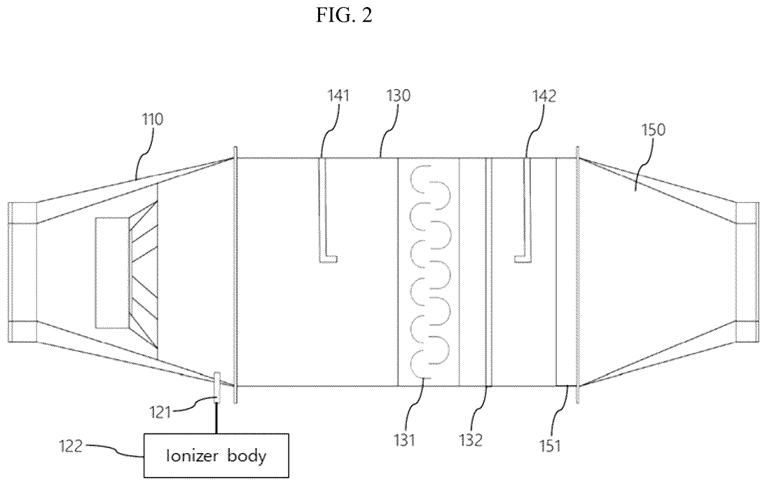

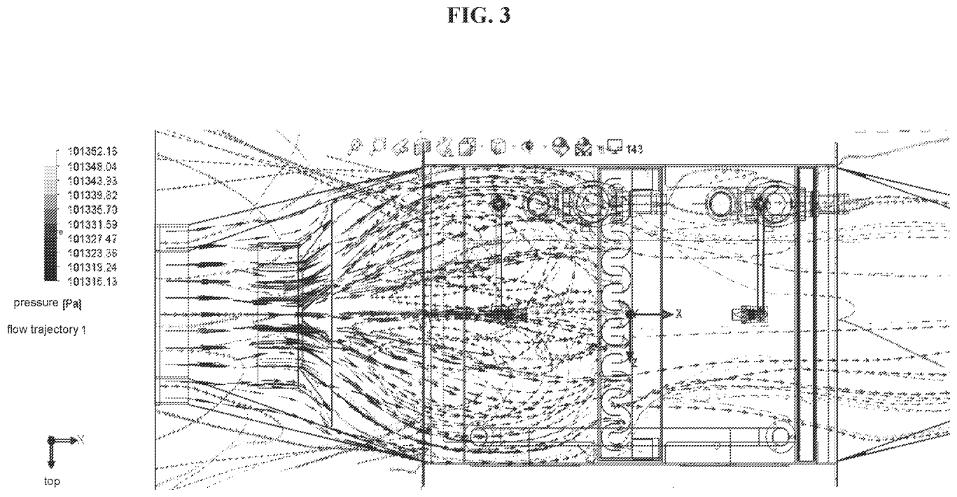

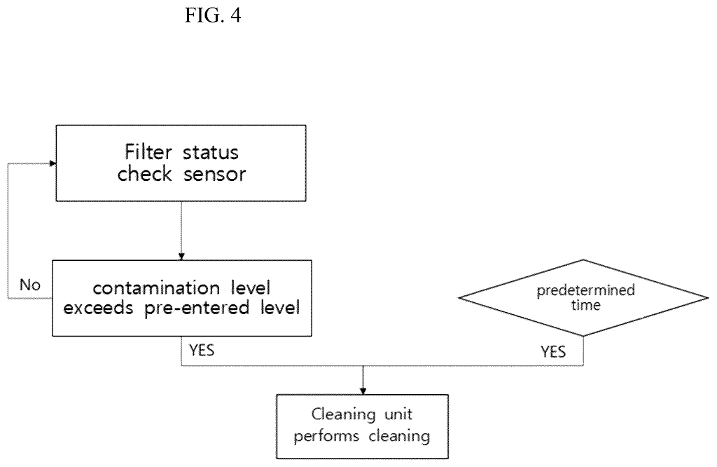

FIG. 1 is an embodiment of an air purification filter device using the X-Ray Ionizer of the present invention, FIG. 2 is an embodiment of the air purification filter device of the present invention, FIG. 3 is an example of flow analysis of the air purification filter device of the present invention, and FIG. 4 shows an example of the cleaning procedure of the cleaning unit of the present invention.

As shown in FIGS. 1 to 4, an air purification filter device using an X-ray ionizer of the present invention comprising:

A first chamber (110) connected to a blower and supplied with contaminated air;

An ionizer unit (120) comprising an ionizer output means (121) formed in a selected portion of the first chamber (110) and providing ions to the contaminated air supplied to the first chamber (110), and
an ionizer body (122) that provides a light source to the ionizer output means (121);

A filter unit (130) comprising a baffle filter (131) formed of a plurality of pillars with a semicircular cross-section and providing a surface for adsorbing fine dust and odor particles from contaminated air charged with positive or negative ions through the ionizer unit (120), and
a mesh filter (132) formed of a metal mesh for adsorbing fine dust and odor particles that have passed through the baffle filter (131);

A cleaning unit (140) comprising a baffle filter cleaning means (141) for cleaning particles adsorbed on the surface of the baffle filter (131) by spraying a cleaning solution onto the baffle filter (131), and
a mesh filter cleaning means (142) that sprays a cleaning solution on the mesh filter (132) to clean particles adsorbed on the surface of the mesh filter (132); and

A second chamber (150) comprising a rear filter (151) that filters foreign substances in the air that have passed through the filter unit (130).

The air purification filter device using an X-ray ionizer of the present invention can receive indoor contaminated air from the first chamber (110).

At this time, the first chamber (110) may include a blower, as shown in FIGS. 2 and 3, and the first chamber (110) can guide a flow of air generated through a plurality of blades included in the blower inside the purification filter device to improve air purification efficiency.

The ionizer unit (120) may provide positive or negative ions to the contaminated air moving inside the first chamber (110).

To explain in more detail, as shown in FIG. 2, one side of the ionizer output means (121) is connected to the ionizer body (122) and the other side is installed in a selected portion of the first chamber (110).

The ionizer output means (121) generates positive and negative ions, and the surface of the contaminated air inside the first chamber (110) may be charged by the generated positive or negative ions. At this time, fine dust and odor particles present in contaminated air may be charged with positive or negative ions.

The ionizer unit (120) can be driven by applying voltage or irradiating light, and the applied voltage can be configured to be selected between 3 and 30 kV.

The ionizer unit (120) can sterilize contaminated air and remove static electricity from contaminated air by generating positive and negative ions.

The ionizer unit (120) does not require airflow supply, does not generate foreign substances, and maintenance of the ionizer unit (120) can be easily performed.

In addition, the ionizer output means (121) can emit X-rays at an angle of 150 degrees, thereby providing positive and negative ions to all areas inside the first chamber (110).

The filter unit (130) comprises the baffle filter (131) formed of a plurality of pillars with a semicircular cross-section and providing a surface for adsorbing fine dust and odor particles from contaminated air charged with positive or negative ions through the ionizer unit (120), and
a mesh filter (132) formed of a metal mesh for adsorbing fine dust and odor particles that have passed through the baffle filter (131).

The filter unit (130) can adsorb fine dust and odor in contaminated air.

The contaminated air is charged with positive or negative ions through the X-rays irradiated from the ionizer output means (121), and when the baffle filter (131) and the mesh filter (132) are grounded, fine dust and odor particles in the contaminated air can be induced and adsorbed to the baffle filter (131) and the mesh filter (132) by electrostatic force.

As shown in the flow analysis of FIG. 3, contaminated air flows into the first chamber (110), is charged by positive or negative ions generated in the ionizer output means (121), is adsorbed and purified while passing through the baffle filter (131) and the mesh filter (132), and the air passing through the baffle filter (131) and the mesh filter (132) moves to the second chamber (150).

At this time, the filter unit (130) may be configured to apply the Coanda Effect to the baffle filter (131).

The Coanda effect is an aerodynamic phenomenon in which a fluid (liquid, gas) with flowing properties flows along a material with a curved surface.

The baffle filter (131) of the present invention is formed with a semicircular cross-section, and the air flowing through it comes into close contact with the baffle filter (131) due to the Coanda effect.

The contaminated air contacts in the baffle filter (131), and fine dust and malodorous particles from the contaminated air can be effectively adsorbed in the baffle filter (131).

At this time, the baffle filter (131) can be replaced with a perforated plate filter, a cyclone filter, a mesh filter, etc., and the mesh filter (132) can be replaced with a baffle filter, a perforated plate filter, a cyclone filter, etc.

The cleaning unit (140) sprays cleaning liquid onto the baffle filter (131) from the baffle filter cleaning means (141) to clean particles adsorbed on the surface, and spray a cleaning solution onto the mesh filter (132) through the mesh filter cleaning means (142) to clean particles adsorbed on the surface.

The baffle filter cleaning means (141) and the mesh filter cleaning means (142) automatically and periodically clean the baffle filter (131) and the mesh filter (132) to optimally maintain contamination level of filter surface, thereby preventing a decrease in adsorption and purification efficiency that may occur due to contamination of the filter.

If the fine dust, oil vapor, and odor particles stuck to the baffle filter (131) and the mesh filter (132) are left without cleaning, the adsorption effect may be weakened and the filtering performance may be reduced.

The present invention allows the surfaces of the baffle filter (131) and the mesh filter (132) to be automatically cleaned for a certain period of time after the end of work or business, so that the best filtering performance can be maintained as if a new filter is installed every day.

In addition, the cleaning unit (140) comprises a filter status check sensor (143) that checks the status of the baffle filter (131) and the mesh filter (132), and
a cleaning control means (144) that analyzes the status of the baffle filter (131) and mesh filter (132) confirmed through the filter status check sensor (143), and controls cleaning through the baffle filter cleaning means (141) and the mesh filter cleaning means (142) when the contamination level exceeds the pre-entered level.

The surface condition of the baffle filter (131) and mesh filter (132) can be checked through the filter condition check sensor (143) of the cleaning unit (140).

At this time, the filter status check sensor (143) may be configured as a sensor for image acquisition or as a method of measuring electrical resistance.

Measured contamination information is compared with information on the degree of contamination requiring predetermined cleaning, and if the measured degree of contamination exceeds the pre-entered degree of contamination, cleaning through the baffle filter cleaning means (141) and the mesh filter cleaning means (142) can be performed.

At this time, the cleaning control means (144) can perform cleaning at predetermined times and at regular intervals.

As an example, the work (business) end time can be input in advance into the cleaning control means (144) and set to automatically perform cleaning when the time has passed.

The baffle filter cleaning means (141) and the mesh filter cleaning means (142) automatically and periodically clean the baffle filter (131) and the mesh filter (132) to optimally maintain the contamination level on the filter surface, thereby preventing a decrease in adsorption and purification efficiency that may occur due to contamination of the filter.

The second chamber (150) can filter foreign substances in the air that have passed through the filter unit (130).

At this time, the second chamber (150) may include a rear filter (151), and the rear filter (151) may be composed of a baffle filter, a mesh filter, a perforated plate filter, a cyclone filter, etc.

### EXPLANATION OF SYMBOLS

| | |
|---|---|
| 110: First chamber | |
| 120: Ionizer unit | |
| 121: Ionizer output means | 122: Ionizer body |
| 130: Filter unit | 131: Baffle Filter |
| 132: Mesh Filter | |
| 140: Cleaning unit | 141: Baffle filter cleaning means |
| 142: Mesh filter cleaning means | 143: Filter status check sensor |
| 144: Cleaning control means | |
| 150: Second chamber | 151: Rear filter |

1. An air purification filter device using an X-ray ionizer comprising:
A first chamber (110) connected to a blower and supplied with contaminated air;
An ionizer unit (120) comprising an ionizer output means (121) formed in a selected portion of the first chamber (110) and providing ions to the contaminated air supplied to the first chamber (110), and
an ionizer body (122) that provides a light source to the ionizer output means (121);
A filter unit (130) comprising a baffle filter (131) formed of a plurality of pillars with a semicircular cross-section and providing a surface for adsorbing fine dust and odor particles from contaminated air charged with positive or negative ions through the ionizer unit (120), and
a mesh filter (132) formed of a metal mesh for adsorbing fine dust and odor particles that have passed through the baffle filter (131);
A cleaning unit (140) comprising a baffle filter cleaning means (141) for cleaning particles adsorbed on the surface of the baffle filter (131) by spraying a cleaning solution onto the baffle filter (131), and
a mesh filter cleaning means (142) that sprays a cleaning solution on the mesh filter (132) to clean particles adsorbed on the surface of the mesh filter (132); and
A second chamber (150) comprising a rear filter (151) that filters foreign substances in the air that have passed through the filter unit (130).

2. An air purification filter device using an X-ray ionizer according to claim 1,
wherein the ionizer unit (120) has a voltage of 3 to 30 kV.

3. An air purification filter device using an X-ray ionizer according to claim 2,
wherein the filter unit (130) is configured to apply the Coanda Effect to the baffle filter (131).

4. An air purification filter device using an X-ray ionizer according to claim 3,
wherein the filter unit (130) is configured to provide a ground connection to the baffle filter (131) and the mesh filter (132).

5. An air purification filter device using an X-ray ionizer according to claim 4,
wherein the baffle filter cleaning means (141) and the mesh filter cleaning means (142) periodically clean the baffle filter (131) and the mesh filter (132).
